(19) United States
(12) Patent Application Publication
(10) Pub. No.: US 2019/0167750 A1
(43) Pub. Date: Jun. 6, 2019

(54) NUTRITIONAL SUPPLEMENTS FOR INCREASING MALE TESTOSTERONE LEVELS

(71) Applicant: Innovus Pharmaceuticals, Inc., San Diego, CA (US)

(72) Inventor: Bassam Damaj, San Diego, CA (US)

(21) Appl. No.: 15/829,799

(22) Filed: Dec. 1, 2017

(51) Int. Cl. A61K 36/67; A61K 31/4415; A61K 33/06; A61K 33/30; A61K 36/41; A61K 31/404; A61K 36/48; A61K 36/068; A61P 5/26; A23L 33/105; A23L 33/15

(52) U.S. Cl. CPC A61K 36/67; A61K 31/4415; A61K 33/06; A61K 33/30; A61K 36/41; A23L 33/15; A61K 36/48; A61K 36/068; A61P 5/26; A23L 33/105; A61K 31/404

(57) ABSTRACT

The invention provides compositions and methods that are effective to boost testosterone levels in a mammal.

### BACKGROUND OF THE INVENTION

Testosterone is a male sex hormone that is important for sexual and reproductive development. The National Institutes of Health regards testosterone as the most important male hormone. Testosterone plays an important role on mind, body and sexual functions in men. Testosterone helps maintain a feeling of wellbeing, maintain healthy bones and muscles, and maintain sex drive and fertility.

By the age of 80, testosterone levels are around pre-pubertal levels in men. To alleviate the unwanted effects of reduced testosterone levels, testosterone boosters are used to promote the natural production of testosterone in the body. Accordingly, testosterone boosters can reduce fat, enhance mood, improve sleep, build muscle, boost sexual performance, reduce cholesterol, promote heart function, and promote lung function.

A previous dietary supplement was designed and sold to increase male testosterone levels. The previous supplement included the following ingredients: vitamin B6, magnesium, zinc, piper nigrum, rhodiola, diindolymethane, tongkat ali, and fenugreek seed extract. It was sold and administered as a capsule containing 15 mg Piper Nigrum (piperine). The recommended dosage was two capsules per day, providing a total of 30 mg Piper Nigrum per day. In spite of the success realized with the previous formulation, there remains a need for additional formulations that provide improved effects.

### SUMMARY OF THE INVENTION

Applicant has determined that the beneficial effects of the previous formulation can be significantly improved by increasing the amount of Piper Nigrum in the capsule from 15 mg to about 20 mg, providing a total of about 40 mg Piper Nigrum per day. Applicant also determined that the addition of Cordyceps Sinesis Mushroom and Ashwagandha Root Extract further improved the effects of the formulation.

Accordingly, in one embodiment the invention provides a composition comprising: Vitamin B6, Magnesium, Zinc, Piper Nigrum (Black Pepper), Zhodiola (Rhodiola Rosea), DIM (Diindolymethane), TongkatAli (Eurycorna Longifolia), Fenugreek Seed Extract, and Cordyceps Sinesis Mushroom.

In another embodiment the invention provides a composition comprising: Vitamin B6, Magnesium, Zinc, Piper Nigrum (Black Pepper), Zhodiola (Rhodiola Rosea), DIM (Diindolymethane), TongkatAli (Eurycorna Longifolia), Fenugreek Seed Extract, and Ashwagandha Root Extract.

In another embodiment the invention provides a composition comprising: Vitamin B6, Magnesium, Zinc, Piper Nigrum (Black Pepper), Zhodiola (Rhodiola Rosea), DIM (Diindolymethane), TongkatAli (Eurycorna Longifolia), Fenugreek Seed Extract, Cordyceps Sinesis Mushroom, and Ashwagandha Root Extract.

In another embodiment the invention provides a unit dosage form comprising at least about 18 mg Piper Nigrum, Vitamin B6, Magnesium, Zinc, Zhodiola (Rhodiola Rosea), DIM (Diindolymethane), TongkatAli (Eurycorna Longifolia), Fenugreek Seed Extract, Cordyceps Sinesis Mushroom, and Ashwagandha Root Extract. In another embodiment the unit dosage form comprises at least about 19 mg Piper Nigrum. In another embodiment the unit dosage form comprises at least about 20 mg Piper Nigrum.

In another embodiment the invention provides a unit dosage form comprising:

| | |
|---|---|
| Vitamin B6 | about 20 mg |
| Magnesium | about 400 mg |
| Zinc | about 30 mg |
| Piper Nigrum (Black Pepper or Bioperine) | about 20 mg |
| Rhodiola (Rhodiola Rosea) | about 114 mg |
| DIM (Diindolymethane) | about 75 mg |
| TongkatAli (Eurycorna Longifolia) | about 25 mg and |
| Fenugreek Seed Extract | about 600 mg |

In another embodiment the unit dosage form comprises about 500 mg Cordyceps Sinesis Mushroom. In another embodiment the unit dosage form comprises about 100 mg Ashwagandha Root Extract.

In another embodiment the invention provides a unit dosage form comprising:

| | |
|---|---|
| Vitamin B6 | 20 mg |
| Magnesium | 400 mg |
| Zinc | 30 mg |
| Piper Nigrum (Black Pepper or Bioperine) | 20 mg |
| Rhodiola (Rhodiola Rosea) | 114 mg |
| DIM (Diindolymethane) | 75 mg |
| TongkatAli (Eurycorna Longifolia) | 25 mg |
| Fenugreek Seed Extract | 600 mg |
| Cordyceps Sinesis Mushroom | 500 mg and |
| Ashwagandha Root Extract. | 100 mg |

In another embodiment the invention provides a method to reduce fat in a mammal comprising administering a composition of the invention or a unit dosage form of the invention to the mammal.

In another embodiment the invention provides a method to enhance mood in a mammal comprising administering a composition of the invention or a unit dosage form of the invention to the mammal.

In another embodiment the invention provides a method to improve sleep in a mammal comprising administering a composition of the invention or a unit dosage form of the invention to the mammal.

In another embodiment the invention provides a method to build muscle in a mammal comprising administering a composition of the invention or a unit dosage form of the invention to the mammal.

In another embodiment the invention provides a method to boost sexual performance in a mammal comprising administering a composition of the invention or a unit dosage form of the invention to the mammal.

In another embodiment the invention provides a method to reduce cholesterol in a mammal comprising administering a composition of the invention or a unit dosage form of the invention to the mammal.

In another embodiment the invention provides a method to promote heart health in a mammal comprising administering a composition of the invention or a unit dosage form of the invention to the mammal.

In another embodiment the invention provides a method to promote lung function in a mammal comprising administering a composition of the invention or a unit dosage form of the invention to the mammal

In another embodiment the invention provides a method to increase sexual function in a mammal comprising administering a composition of the invention or a unit dosage form of the invention to the mammal.

In another embodiment the invention provides a method to support natural testosterone levels in a mammal comprising administering a composition of the invention or a unit dosage form of the invention to the mammal.

In another embodiment the invention provides a method to improve muscular strength in a mammal comprising administering a composition of the invention or a unit dosage form of the invention to the mammal.

In another embodiment the invention provides a method to improve stamina in a human comprising administering a composition of the invention or a unit dosage form of the invention to the human.

In another embodiment the invention provides a method to improve mental performance in a human comprising administering a composition of the invention or a unit dosage form of the invention to the human.

### DETAILED DESCRIPTION

Vitamin B6 helps the body make neurotransmitters and supports production of serotonin and norepinephrine which impact mood. Low levels of serotonin are associated with depression. According to the Office of Dietary Supplements, vitamin B6 plays a key role in the production of energy. It is essential to the metabolism of hemoglobin, which transport oxygen through the blood and it helps convert amino acids into niacin and assists in the regulation of blood sugar. Low vitamin B status is associated with poor immune system functioning in elderly individuals. It also improves the metabolism in tissues of the brain. Because it is water soluble, it cannot be stored in the body and needs to come from daily intake.

Magnesium activates enzymes for neuromuscular contractions, and cellular energy as well as relaxation of muscles. It helps regulate levels of calcium, copper, zinc, potassium, vitamin D, and other important nutrients in the body. Inadequate magnesium may reduce serotonin levels. It is also an important component for the biosynthesis of proteins, as well as for ensuring the processes of excitability and contractility in the nerve elements of muscle tissue. Magnesium is also a regulator of the passing of redundant nerve impulses from the brain to peripheral nerves and muscles, prevents excessive spasms (involuntary muscle contractions), including sexual system.

Zinc is a powerful antioxidant which is important for the maintenance of hormones and normal testosterone levels. It is a Tier 3 supplement for prostatitis, prostate cancer and BPH.

Studies indicate a role in testosterone, sperm formation/mobility, and DNA damage repair.

Rhodiola Rosea has been clinically shown to improve mental performance. Studies have also demonstrated that it promotes an increase in pulmonary ventilation and physical capacities.

Diindolymethane (DIM) is thought to block conversion of testosterone in the body by suppressing the actions of aromatase to convert testosterone to estradiol resulting in more free testosterone in the system.

Eurycorna Longifolia (Tongkat Ali) supports total and free testosterone. It also supports healthy cortisol levels, enhances energy, decreases fat mass, and functions as an antioxidant. Traditionally it has been consumed as an aphrodisiac for increasing testosterone levels, increasing the production of sperm, increasing vasodilation, improving erectile function, and reducing fat mass

Fenugreek supports testosterone levels and sexual function. It contains soluble steroidal saponins, specifically furostanol glycosides responsible for complexing cholesterol in the cell membrane. Clinical studies in adult and aging men have shown sexual function improvement, including morning erections and frequency of sexual activity, increased total serum testosterone and free testosterone levels, reduction in symptoms of possible androgen deficiency, a positive effect on sexual health and quality of life, and anabolic and androgenic activity.

Cordyceps is a genus of ascomycete fungi includes about 400 species. Most Cordyceps species are endoparasitoids, parasitic mainly on insects and other arthropods. Cordyceps Sinesis was shown in 2007 to be unrelated to most of the rest of the members of the genus. As a result, it was placed in a new family and renamed Ophiocordyceps sinensis. Cordyceps Sinesis is commercially available from a variety of sources.

Bioperine® is a patented absorption enhancer, obtained from black pepper fruits (Piper nigrum). Bioperine® helps the body absorb the amino acids (see U.S. Pat. No. 5,536,506 and European Patent EP0810868B1). Bioperine® (extract) inhibits human CYP3A4 and P-glycoprotein enzymes. By inhibiting certain enzymes BioPerine® (extract) may alter the effectiveness of certain medications by increasing bioavailability. Bioperine is Generally Recognized As Safe (GRAS). Bioperine® (CAS Reg. No. 94-62-2) is named as 1-piperylpiperidine; 5-(1,3-benzodioxol-5-yl)-1-(piperidin-1-yl)penta-2,4-dien-1-one; and (2E,4E)-5-(1,3-benzodioxol-5-yl)-1-(piperidin-1-yl)penta-2,4-dien-1-one, and has the structure:

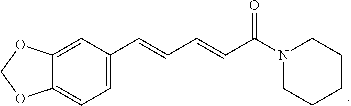 [SMILES: O=C(/C=C/C=C/c1ccc2c(c1)OCO2)N1CCCCC1]

1-Piperylpiperidine is commercially available from a variety of sources.

As used herein, the term “about” has its generally accepted meaning. In one embodiment, the term about means ±10% of the associated value. For example, about 100 mg means 100 mg±10 mg. In one embodiment, the term about means ±5% of the associated value. For example, about 100 mg means 100 mg±5 mg. In one embodiment, the term about means ±2% of the associated value. For example, about 100 mg means 100 mg±2 mg. In one embodiment, the term about means ±1% of the associated value. For example, about 100 mg means 100 mg±1 mg.

The present compositions may be systemically administered, e.g., orally, in combination with a pharmaceutically acceptable vehicle such as an inert diluent or an assimilable edible carrier. They may be enclosed in hard or soft-shell gelatin capsules, may be compressed into tablets, or may be incorporated directly with the food of the patient's diet. For oral therapeutic administration, the active compounds may be combined with one or more excipients and used in the form of ingestible tablets, buccal tablets, troches, capsules, elixirs, suspensions, syrups, wafers, and the like.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. In one embodiment of the invention the compositions of the invention or the unit dosage forms of the invention comprise microcrystalline cellulose, silicon dioxide, and magnesium stearate. In one embodiment of the invention the composition or unit dosage form is formulated as a capsule.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A composition comprising:
Vitamin B6
Magnesium
Zinc
Piper Nigrum (Black Pepper or Bioperine)
Rhodiola (Rhodiola Rosea)
DIM (Diindolymethane)
TongkatAli (Eurycorna Longifolia)
Fenugreek Seed Extract and
Cordyceps Sinesis Mushroom.

2. A composition comprising:
Vitamin B6
Magnesium
Zinc
Piper Nigrum (Black Pepper or Bioperine)
Rhodiola (Rhodiola Rosea)
DIM (Diindolymethane)
TongkatAli (Eurycorna Longifolia)
Fenugreek Seed Extract and
Ashwagandha Root Extract

3. A composition comprising:
Vitamin B6
Magnesium
Zinc
Piper Nigrum (Black Pepper or Bioperine)
Rhodiola (Rhodiola Rosea)
DIM (Diindolymethane)
TongkatAli (Eurycorna Longifolia)
Fenugreek Seed Extract
Cordyceps Sinesis Mushroom and
Ashwagandha Root Extract

4. A unit dosage form comprising at least about 18 mg Piper Nigrum,
Vitamin B6
Magnesium
Zinc
Rhodiola (Rhodiola Rosea)
DIM (Diindolymethane)
TongkatAli (Eurycorna Longifolia)
Fenugreek Seed Extract
Cordyceps Sinesis Mushroom and
Ashwagandha Root Extract.

5. The unit dosage form of claim 4 that comprises at least about 19 mg Piper Nigrum.

6. The unit dosage form of claim 4 that comprises at least about 20 mg Piper Nigrum.

7. The unit dosage form of claim 6 that comprises about 500 mg Cordyceps Sinesis Mushroom.

8. The unit dosage form of claim 6 that comprises about 100 mg Ashwagandha Root Extract.

9. A unit dosage form comprising:

| | |
|---|---|
| Vitamin B6 | about 20 mg |
| Magnesium | about 400 mg |
| Zinc | about 30 mg |
| Piper Nigrum (Black Pepper or Bioperine) | about 20 mg |
| Rhodiola (Rhodiola Rosea) | about 114 mg |
| DIM (Diindolymethane) | about 75 mg |
| TongkatAli (Eurycorna Longifolia) | about 25 mg and |
| Fenugreek Seed Extract | about 600 mg |

10. The unit dosage form of claim 9 that further comprises about 500 mg Cordyceps Sinesis Mushroom.

11. The unit dosage form of claim 9 that further comprises about 100 mg Ashwagandha Root Extract.

12. The unit dosage form of claim 10 that further comprises about 100 mg Ashwagandha Root Extract.

13. The unit dosage form of claim 12 wherein the term about, at each occurrence, means ±5% of the associated value.

14. A unit dosage form comprising:

| | |
|---|---|
| Vitamin B6 | 20 mg |
| Magnesium | 400 mg |
| Zinc | 30 mg |
| Piper Nigrum (Black Pepper or Bioperine) | 20 mg |
| Rhodiola (Rhodiola Rosea) | 114 mg |
| DIM (Diindolymethane) | 75 mg |
| TongkatAli (Eurycorna Longifolia) | 25 mg |
| Fenugreek Seed Extract | 600 mg |
| Cordyceps Sinesis Mushroom | 500 mg and |
| Ashwagandha Root Extract. | 100 mg |

15. The unit dosage form of claim 14 that further comprises microcrystalline cellulose, silicon dioxide, and magnesium stearate.

16. The unit dosage form of claim 15 that is formulated as a capsule.

17. A method to reduce fat, build muscle, improve muscular strength, or improve stamina in a mammal comprising administering a unit dosage form of claim 16 to the mammal.

18. A method to enhance mood or improve sleep in a mammal comprising administering a unit dosage form of claim 16 to the mammal.

19. A method to boost sexual performance or support natural testosterone levels in a mammal comprising administering a unit dosage form of claim 16 to the mammal.

20. A method to reduce cholesterol or promote heart health in a mammal comprising administering a unit dosage form of claim 16 to the mammal.

21. A method to improve mental performance in a mammal comprising administering a unit dosage form of claim 16 to the mammal.
